(19) United States
(12) Patent Application Publication
(10) Pub. No.: US 2016/0021904 A1
(43) Pub. Date: Jan. 28, 2016

(54) DEVICES AND KITS FOR PRODUCTION OF PERSONALIZED, CONCENTRATED HUMAN MILK COMPOSITIONS, AND METHODS OF MAKING AND USING SAME

(71) Applicant: Nestec S.A., Vevey (CH)

(72) Inventor: Lesley Keane VERDI, Chester, NJ (US)

(21) Appl. No.: 14/809,446

(22) Filed: Jul. 27, 2015

Related U.S. Application Data
(60) Provisional application No. 62/029,593, filed on Jul. 28, 2014.

(51) Int. Cl. A23C 9/20; B01D 61/22; B01D 61/18; G01N 33/487; A23C 9/142

(52) U.S. Cl. CPC A23C 9/206; G01N 33/487; A23C 9/1422; B01D 61/18; B01D 61/22; B01D 2313/90

(57) ABSTRACT

Kits and devices may be used to produce a personalized, concentrated human milk composition for an infant from expressed milk from the infant's mother. Methods of production and use thereof are also disclosed.

### BACKGROUND

Premature babies and low-birth-weight infants often need more concentrated nutrition due to their smaller, less developed digestive systems. Current strategies in the Neonatal Intensive Care Unit (NICU) include the use of cow milk-based human milk fortifier (HMF), which is added to human milk. Cow milk-based HMF provides significantly more protein and fat than human milk alone.

However, experts concur that delivery of species-specific nutrition to these infants is best, as it mitigates the risks of introducing allergens and as it provides valuable microflora and micronutrients needed by the species. Recent advancements have been made that allow for the production of human milk-based HMF from multiple donor milk supplies. However, there are some risks of transmitting disease when donor milk is used, so the human donor milk has to be sterilized and therefore may not have as many valuable microflora or micronutrients therein.

Therefore, there is a need in the art for new and improved methods of providing human milk fortifiers for premature and/or low-birth-weight infants that overcome the disadvantages and defects of the prior art, including the uses of cow milk-based HMF as well as human donor milk-based HMF. It is to such methods for producing and using human milk fortifiers, and especially personalized human milk fortifiers specifically produced from breast milk obtained from an individual infant's mother, as well as kits and devices related thereto, that the presently disclosed inventive concept(s) is directed.

### BRIEF DESCRIPTION OF THE DRAWINGS

FIG. 1 is a schematic flow chart illustrating one non-limiting example of a method constructed in accordance with the presently disclosed inventive concept(s).

### DETAILED DESCRIPTION

Before explaining at least one embodiment of the presently disclosed inventive concept(s) in detail, it is to be understood that the presently disclosed inventive concept(s) is not limited in its application to the details of construction and the arrangement of the components or steps or methodologies set forth in the following description or illustrated in the drawings. The presently disclosed inventive concept(s) is capable of other embodiments or of being practiced or carried out in various ways. Also, it is to be understood that the phraseology and terminology employed herein is for the purpose of description and should not be regarded as limiting.

Unless otherwise defined herein, technical terms used in connection with the presently disclosed inventive concept(s) shall have the meanings that are commonly understood by those of ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the devices, kits, and/or methods disclosed or otherwise contemplated herein can be made and executed without undue experimentation in light of the present disclosure. While the devices, kits, and methods of the presently disclosed inventive concept(s) have been described in terms of particular embodiments, it will be apparent to those of ordinary skill in the art that variations may be applied to the devices, kits, and/or methods as well as in the steps and/or in the sequence of steps of the methods described herein without departing from the concept, spirit, and scope of the presently disclosed inventive concept(s). All such similar substitutes and modifications apparent to those of ordinary skill in the art are deemed to be within the spirit, scope, and concept of the presently disclosed inventive concept(s).

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example, but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, and/or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The terms “baby” and “infant” are used interchangeably herein and will be understood to include, but are not limited to, newborns, neonates, premature babies/infants (also referred to as preterm babies/infants or preemies), and low-birth-weight babies/infants.

The terms “human milk” and “breast milk” are used interchangeably herein and will be understood to include any form of milk, including, but not limited to, colostrum, foremilk, and hindmilk.

The terms “personalized human milk fortifier,” “personalized, fortified concentrated human milk,” and “personalized, concentrated human milk composition” are used interchangeably herein and will be understood to refer to a product formed of concentrated breast milk, wherein the breast milk is obtained from a mother of an infant to which the product is to be delivered.

The term “ultrafiltration” as used herein refers to a membrane-based filtration process that is use in industrial and biological settings to separate, purify, and concentrate macromolecular solutions. The term “selective ultrafiltration” refers to the selective concentration of one or more components of a fluid, and may further involve the separation and purification of said concentrated component(s) from one or more undesirable components. The term “selective ultrafiltration device” refers to a device capable of performing said selective separation, purification, and/or concentration of one or more components (i.e., nutrients and/or micronutrients) of a fluid, such as but not limited to, breast milk.

The term “associate” as used herein will be understood to refer to the direct or indirect connection of two or more items.

Turning now to the presently disclosed inventive concept(s), provided herein are devices, kits, and methods related to the need for premature and/or low-birth-weight babies to receive their own mother's milk. In certain embodiments, the presently disclosed inventive concept(s) is directed to devices, kits, and methods that allow each mother of a premature and/or low-birth-weight infant to concentrate her own milk and feed it to her infant, for example but not by way of limitation, during the first fragile days of life in the NICU. The benefits of these devices, kits, and methods include, but are not limited to: (1) emotional benefits—parents will have less concerns, as the personalized human milk fortifier is delivering to the infant only his/her own mother's milk that has been directly expressed and concentrated; and (2) physical benefits—the infants receive the goodness of their own mother's milk (which may even be provided at a specific concentration needed for their specific height, weight, and/or growth stage). Another advantage over the prior art is that there is no need for sterilization of the milk before feeding, and thus more nutrients/micronutrients/microflora are retained in the milk. In addition to providing a superior and personalized product over the prior art HMFs that are cow milk-based or human donor milk-based, the presently disclosed devices, kits, and methods provide a more cost effective product as well.

The presently disclosed inventive concept(s) possesses many benefits over the prior art. The goodness of a mother's own milk is provided to her premature and/or low-birth-weight infant by concentrating the milk to make personalized human milk fortifier that meets the personal nutritional needs of the premature and/or low-birth-weight infant while retaining the valuable microflora and nutrients/micronutrients present in the mother's own breast milk. There is no risk of donor milk transmitted diseases. In addition to providing additional probiotics and other beneficial microflora that are present in the expressed milk, the presently disclosed inventive concept(s) allows for selective concentration of specific nutrient(s) (such as, but not limited to, protein, fat, and carbohydrate); specific peptide(s); specific fatty acid(s); and/or specific micronutrient(s) (such as, but not limited to, calcium, phosphorous, potassium, sodium, and chloride) present in the expressed milk. Another advantageous feature of the presently disclosed inventive concept(s) is the selective removal of undesirable foreign substances from breast milk, such as but not limited to, nicotine, alcohol, medication(s), pharmaceutical by-product(s), pharmaceutical metabolite(s), chemical(s), certain bacteria, virus(es), and combinations thereof.

Certain embodiments of the presently disclosed inventive concept(s) are directed to devices that are used in the production of personalized human milk fortifiers. These devices include, but are not limited to, selective concentration devices that are capable of concentrating at least one nutrient and/or at least one micronutrient present in expressed breast milk from a mother of the infant. These devices will be described in further detail herein below.

Certain embodiments of the presently disclosed inventive concept(s) are directed to kits useful for producing personalized human milk fortifiers. The kit may contain any combination of the below-described components/devices. In addition, the kit may further contain other component(s) and/or device(s) for performing any of the methods/processes described or otherwise contemplated herein. The nature of these additional component(s)/device(s) will depend upon the particular method/process format, and identification thereof is well within the skill of one of ordinary skill in the art.

In one embodiment of the presently disclosed inventive concept(s), a kit is provided for producing personalized human milk fortifier for an infant. The kit may include a selective concentration device that is capable of concentrating at least one nutrient and/or at least one micronutrient present in expressed breast milk from a mother of the infant. The selective concentration device may be capable of concentrating a plurality of nutrients and/or micronutrients. The kit may also include at least one graduated container (such as, but not limited to, a graduated cylinder) that is capable of receiving the expressed breast milk and that is capable of providing a measurement of a volume for the expressed breast milk. The graduated container(s) is also capable of association with the selective concentration device for concentration of the breast milk disposed therein to produce the personalized human milk fortifier.

The kit may further include an analyzer for determining a concentration of at least one nutrient and/or at least one micronutrient present in expressed breast milk. For example, but not by way of limitation, the analyzer may determine a concentration for each of a plurality of nutrients and/or micronutrients present in expressed breast milk. Calculation of specific concentrations present in each sample of breast milk is quite useful, since macronutrients in human milk can vary by day and even by the time of day.

Non-limiting examples of nutrients present in expressed breast milk for which a concentration may be determined (and for which concentration thereof may be desired) include protein, fat, and carbohydrate. Non-limiting examples of micronutrients present in expressed breast milk for which a concentration may be determined (and for which concentration thereof may be desired) include calcium, phosphorous, potassium, sodium, and chloride.

In addition, the kit may further include a device that calculates a desired level of concentration of the breast milk, wherein the calculation is based upon at least two values: (1) a concentration of at least one nutrient and/or at least one micronutrient present in expressed breast milk, and (2) at least one infant factor. Any infant factors that may affect the amount of nutrients/micronutrients required for the infant and/or the volume in which the nutrients/micronutrients should be delivered may be utilized in accordance with the presently disclosed inventive concept(s). Examples of infant factors include, but are not limited to, an infant's height, an infant's weight, an infant's nutritional needs, an infant's fluid volume capacity, an infant's growth curve, and combinations thereof.

The device may calculate the desired level of concentration of the breast milk based upon a concentration of each of a plurality of nutrients and/or micronutrients present in expressed breast milk and a plurality of infant factors. When multiple nutrients/micronutrients/infant factors are considered, the desired level of concentration may be calculated as a desired concentration of one or more of the plurality of nutrients and/or micronutrients. Alternatively, the desired level of concentration may not be based on a specific value for a single nutrient/micronutrient, but may rather be calculated as a desired ratio of two of the nutrients/micronutrients to one another.

Any device capable of calculating one or more desired concentration values (or ratios) as described herein above—and as based on the input of multiple variables—may be utilized in accordance with the presently disclosed inventive concept(s). Non-limiting examples of devices that may be utilized include simple, manually manipulated devices (such as, but not limited to, slide rule-type devices, nomogram-type devices, sliding window(s)-type devices, as well as any other type of chart and/or diagram that requires manual manipulation to determine a calculated value) as well as logic devices (such as, but not limited to, software and/or hardware—type devices, such as a microprocessor, microcontroller, application specific integrated circuit, field programmable gate array, and the like).

For example, but not by way of limitation, the calculated desired concentration value(s) may be in a range of from about 1.1× to about 10×. In particular, non-limiting examples the calculated desired concentration value(s) may be in any range having (i) a low end value of any of about 1.1×, about 1.2×, about 1.3×, about 1.4×, about 1.5×, about 1.6×, about 1.7×, about 1.8×, about 1.9×, about 2.0×, about 2.1×, about 2.2×, about 2.3×, about 2.4×, about 2.5×, about 2.6×, about 2.7×, about 2.8×, about 2.9×, about 3.0×, about 3.1×, about 3.2×, about 3.3×, about 3.4×, about 3.5×, about 3.6×, about 3.7×, about 3.8×, about 3.9×, about 4.0×, about 4.1×, about 4.2×, about 4.3×, about 4.4×, about 4.5×, about 4.6×, about 4.7×, about 4.8×, about 4.9×, about 5.0×, about 5.1×, about 5.2×, about 5.3×, about 5.4×, about 5.5×, about 5.6×, about 5.7×, about 5.8×, about 5.9×, about 6.0×, about 6.1×, about 6.2×, about 6.3×, about 6.4×, about 6.5×, about 6.6×, about 6.7×, about 6.8×, about 6.9×, about 7.0×, about 7.1×, about 7.2×, about 7.3×, about 7.4×, about 7.5×, about 7.6×, about 7.7×, about 7.8×, about 7.9×, about 8.0×, about 8.1×, about 8.2×, about 8.3×, about 8.4×, about 8.5×, about 8.6×, about 8.7×, about 8.8×, about 8.9×, about 9.0×, and the like; and (ii) a high end value of any of about 2.0×, about 2.1×, about 2.2×, about 2.3×, about 2.4×, about 2.5×, about 2.6×, about 2.7×, about 2.8×, about 2.9×, about 3.0×, about 3.1×, about 3.2×, about 3.3×, about 3.4×, about 3.5×, about 3.6×, about 3.7×, about 3.8×, about 3.9×, about 4.0×, about 4.1×, about 4.2×, about 4.3×, about 4.4×, about 4.5×, about 4.6×, about 4.7×, about 4.8×, about 4.9×, about 5.0×, about 5.1×, about 5.2×, about 5.3×, about 5.4×, about 5.5×, about 5.6×, about 5.7×, about 5.8×, about 5.9×, about 6.0×, about 6.1×, about 6.2×, about 6.3×, about 6.4×, about 6.5×, about 6.6×, about 6.7×, about 6.8×, about 6.9×, about 7.0×, about 7.1×, about 7.2×, about 7.3×, about 7.4×, about 7.5×, about 7.6×, about 7.7×, about 7.8×, about 7.9×, about 8.0×, about 8.1×, about 8.2×, about 8.3×, about 8.4×, about 8.5×, about 8.6×, about 8.7×, about 8.8×, about 8.9×, about 9.0×, about 9.1×, about 9.2×, about 9.3×, about 9.4×, about 9.5×, about 9.6×, about 9.7×, about 9.8×, about 9.9×, about 10×, and the like.

In certain embodiments, the kit may further include at least one feeding apparatus in which the personalized human milk fortifier may be disposed and that may be used for delivery of the personalized human milk fortifier to the infant. In one particular, non-limiting embodiment, the feeding apparatus may be a feeding syringe, so as to avoid nipple confusion (especially if the mother is breastfeeding or plans to breastfeed upon discharge).

In certain embodiments, the selective concentration device may be a selective ultrafiltration device and thus may be capable of removing at least one undesirable substance from the expressed breast milk. In other embodiments, the kit may include a separate filtration device for removing at least one undesirable substance from the expressed breast milk. Examples of undesirable substances that may potentially be present in breast milk include, but are not limited to, nicotine, alcohol, medications, pharmaceutical by-products, pharmaceutical metabolites, chemicals, certain bacteria, viruses, and combinations thereof.

The kits of the presently disclosed inventive concept(s) may further include one or more sets of instructions. The instructions may explain how to use the kit, how to deliver the personalized human milk fortifier to the infant, and/or how to store the personalized human milk fortifier. Non-limiting, exemplary storage periods that may be provided in the instructions include storage under refrigerated conditions lesser than or equal to 35° F. (1.6° C.), plus or minus 5° F. or 5° C., for a period of up to five days and storage under frozen conditions lesser than or equal to 0° F. (−18° C.), plus or minus 5° F. or 5° C., for a period of up to six months.

Any type of format capable of conveying the desired information (and/or directing a user's attention to a location where said information can be found) may be utilized as the instructions described or otherwise contemplated herein. Non-limiting examples of formats in which the instructions may be provided include written wording and/or pictorial drawings, a website address, a bar code (such as but not limited to, a QR code) that is readable by an imaging device/code reader, combinations thereof, and the like.

Any of the kits described herein above or otherwise contemplated herein may be used with any of the methods described or otherwise contemplated herein below.

Certain other embodiments of the presently disclosed inventive concept(s) are directed to a method of producing a personalized human milk fortifier for an infant. In the method, expressed breast milk is obtained from a mother of the infant, and a portion thereof is analyzed to determine a concentration of at least one nutrient and/or at least one micronutrient present therein. A desired level of concentration of the expressed breast milk is then calculated based upon: (1) the concentration of the at least one nutrient and/or at least one micronutrient present in the expressed breast milk, and (2) at least one infant factor (as described in detail herein above). The expressed breast milk is then exposed to a selective concentration device that concentrates the at least one nutrient and/or the at least one micronutrient (that was analyzed as described herein above) to the desired level of concentration as calculated above, thereby producing the personalized human milk fortifier that is specific for the infant and obtained directly from the milk of the infant's mother.

The method may further include the step of administering at least a portion of the personalized human milk fortifier to the infant. Alternatively and/or in addition thereto, the method may further include the step of storing the personalized human milk fortifier under refrigerated or frozen conditions for a period of time.

In particular, non-limiting embodiments of the presently disclosed inventive concept(s), a concentration of a plurality of nutrients and/or micronutrients present is the expressed breast milk is determined; in this embodiment, the selective concentration device concentrates the plurality of nutrients and/or micronutrients as described above. When the calculated level of concentration for the breast milk is based upon a concentration of each of a plurality of nutrients and/or micronutrients present in the expressed breast milk as well as a plurality of infant factors, the calculated level of concentration may include: (i) a desired specific concentration of one or more of the plurality of nutrients and/or micronutrients, and/or (ii) a desired ratio of two of the nutrients/micronutrients to one another.

In certain embodiments, the method may further include the step of filtering the expressed breast milk to remove at least one undesirable substance (such as, but not limited to, nicotine, alcohol, medications, pharmaceutical by-products, pharmaceutical metabolites, chemicals, certain bacteria, viruses, and combinations thereof) from the expressed breast milk. This filtration step may be performed by the selective concentration device if the selective concentration device is a selective ultrafiltration device. Alternatively, a separate filtration device may be utilized to perform this step.

The methods of the presently disclosed inventive concept(s) may be performed in the absence of any sterilization steps, thus retaining substantially all desired components of the expressed breast milk.

The combinations of method steps described herein above may be performed simultaneously or wholly or partially sequentially. In addition, the exemplary sequences of method steps provided herein above are for the purposes of illustration only; it will be understood that the individual steps, as well as the particular order of steps, may vary, and the sequence of steps may be performed in any order, so long as the materials and packages described herein are capable of functioning in accordance with the presently disclosed inventive concept(s).

### EXAMPLES

Examples are provided herein below. However, the presently disclosed inventive concept(s) is to be understood to not be limited in its application to the specific experimentation, results, and procedures described herein after. Rather, the Examples are simply provided as one of various embodiments and are meant to be exemplary, not exhaustive.

### Example 1

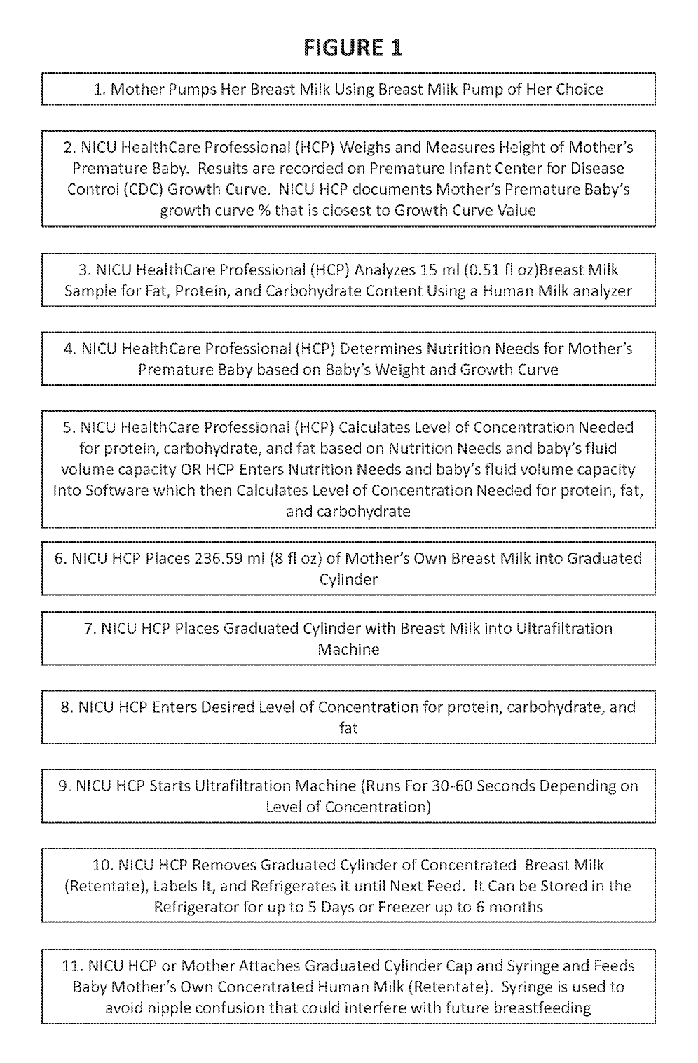

FIG. 1 is a schematic flow chart illustrating one embodiment of a method constructed in accordance with the presently disclosed inventive concept(s).

### Example 2

In one example of a method constructed in accordance with the presently disclosed inventive concept(s), a small scale, selective ultrafiltration procedure is used so that a mother's own expressed milk is concentrated to a level needed based on the infant's nutritional needs and daily liquid volume capacity. A human milk analyzer can be used just prior to concentrating the milk, since macronutrients in human milk vary by day and daypart, to determine the exact level of concentration. A software program can be provided, and a healthcare professional (HCP) can enter in the results of the human milk analysis and the desired nutrition and/or liquid volume (i.e., in milliliters). The software program may calculate the level of concentration needed.

If the concentrated milk is not needed immediately, it can be refrigerated or frozen for a period of time. For example but not by way of limitation, the concentrated milk can be refrigerated for up to five days lesser than or equal to 35° F. (1.6° C.), plus or minus 5° F. or 5° C. or frozen lesser than or equal to 0° F. (−18° C.), plus or minus 5° F. or 5° C., for up to six months. Defrosting should be done overnight in the fridge or in a warm water bath for no longer than 15 minutes. Any unused milk should be discarded after warming in water bath.

TABLE I
Selective Concentration Example

| | Pumped | Pumped | Selective Concentration |
|---|---|---|---|
| | Breast | Breast Milk + | needed for Concentrated |
| | Milk | Liquid HMF | Breast Milk |
| Energy kcal | | | |
| Protein (g) | 1.6 | 3.2 | 2X |
| Fat (g) | 4.2 | 4.8 | 1.1X |
| Carbohydrate (g) | 7 | 6.6 | 1X |
| Calcium (mg) | 28 | 118 | 4.2X |
| Phosphorous (mg) | 14 | 65 | 4.6X |

TABLE II
Selective Concentration Example - 1.5 kg baby

| | Daily Nutrient | | | | Concentration of |
|---|---|---|---|---|---|
| | Requirement for | | | | preterm breast milk |
| | preterm infant weighing | | Preterm Breast milk* | | needed to meet |
| | 1.5 kg (AAP Committee | | (AAP, | | preterm infant daily |
| 1.5 kg baby | of Nutrition, 2013) | | pediatriccareonline.org) | | requirement |
| Volume ml/day | 240 | | 240 | | na |
| Energy | 180.0 | Kcal/day | 160.8 | Kcal/day | 1.12X |
| Protein | 6.3 | g/day | 3.9 | g/day | 1.62X |
| Carbohydrate | 25.5 | g/day | 15.9 | g/day | 1.60X |
| Fat | 10.8 | g/day | 9.3 | g/day | 1.16X |
| Calcium | 330.0 | mg/day | 59.5 | mg/day | 5.54X |
| Phosphorous | 210.0 | mg/day | 30.7 | mg/day | 6.84X |

*Values can vary based on maternal nutrition
Calculations assume volume is held constant at 240 ml/day for both preterm breast milk and concentrated preterm breast milk.

### Example 3

A non-limiting example of a kit constructed in accordance with the presently disclosed inventive concept(s) includes the following components: (a) a small scale ultrafiltration machine for liquid volumes between 2-16 fluid ounces; (b) graduated cylinders—small (2-8 oz.) cylinder(s) and/or large (8-16 oz.) cylinder(s); (c) graduated cylinder caps for refrigeration or frozen storage of the personalized human milk fortifier in the graduated cylinder(s); and (d) a software program (or other device) to calculate the level of human milk concentration needed by ultrafiltration.

Other components that may be present in the kit include the following: (a) a breast milk analyzer for determining content of various nutrients (i.e., protein, fat, carbohydrate, etc.) and/or micronutrients (i.e., calcium, phosphorous, etc.) present in the breast milk; and (b) feeding syringe(s).

For example, approximately 8 oz. of fluid milk is poured into a graduated cylinder and placed into an ultrafiltration unit. The level of concentration is entered, and the start button on the ultrafiltration unit is pushed. Within seconds, the mother's milk is concentrated and is ready to be fed to the infant by any known method. One specific, non-limiting feeding method uses a syringe, so as to avoid nipple confusion (especially if the mother is breastfeeding or plans to breastfeed upon discharge).

Thus, in accordance with the presently disclosed inventive concept(s), there have been provided methods of producing a personalized human milk fortifier, as well as devices and kits related to same, that fully satisfy the objectives and advantages set forth hereinabove. Although the presently disclosed inventive concept(s) has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the presently disclosed inventive concept(s). Changes may be made in the construction and the operation of the various components, elements, and assemblies described herein, as well as in the steps or the sequence of steps of the methods described herein, without departing from the spirit and scope of the presently disclosed inventive concept(s).

What is claimed is:

1. A kit for producing a personalized, concentrated human milk composition for an infant, the kit comprising:
at least one analyzer for determining a concentration of at least one nutrient and/or at least one micronutrient present in the expressed breast milk from the mother;
at least one selective concentration device capable of concentrating at least one nutrient and/or at least one micronutrient present in expressed breast milk from a mother of the infant; and
at least one graduated container capable of receiving the expressed breast milk and capable of measuring a volume of the expressed breast milk, the at least one graduated container being capable of association with the selective concentration device for concentration of the breast milk disposed therein to produce the personalized, concentrated human milk composition.

2. The kit of claim 1, wherein the selective concentration device is capable of concentrating a plurality of nutrients and/or micronutrients.

3. The kit of claim 1, wherein the analyzer determines a concentration for each of a plurality of nutrients and/or micronutrients present in the expressed breast milk.

4. The kit of claim 1, further comprising at least one device that calculates a desired level of concentration of the breast milk based upon a concentration of at least one nutrient and/or at least one micronutrient present in the expressed breast milk and at least one infant factor selected from the group consisting of the infant's height, the infant's weight, the infant's nutritional needs, the infant's fluid volume capacity, the infant's growth curve, and combinations thereof.

5. The kit of claim 4, wherein the device calculates a desired level of concentration of the breast milk based upon a concentration of each of a plurality of nutrients and/or micronutrients present in the expressed breast milk and a plurality of infant factors, and wherein the calculated level of concentration is defined as:
(i) a desired concentration of one or more of the plurality of nutrients and/or micronutrients; and/or
(ii) a desired ratio of two of the nutrients/micronutrients to one another.

6. The kit of claim 1, wherein each nutrient is selected from the group consisting of protein, fat, and carbohydrate, and wherein each micronutrient is selected from the group consisting of calcium, phosphorous, potassium, sodium, and chloride.

7. The kit of claim 1, further comprising at least one feeding apparatus in which the personalized, concentrated human milk composition may be disposed for delivery to the infant.

8. The kit of claim 1, wherein the selective concentration device is further defined as a selective ultrafiltration device.

9. The kit of claim 8, wherein the selective ultrafiltration device is capable of removing at least one undesirable substance from the expressed breast milk.

10. The kit of claim 9, wherein the at least one undesirable substance is selected from the group consisting of nicotine, alcohol, medications, pharmaceutical by-products, pharmaceutical metabolites, chemicals, certain bacteria, viruses, and combinations thereof.

11. The kit of claim 1, further comprising at least one filtration device for removing at least one undesirable substance from the expressed breast milk.

12. The kit of claim 11, wherein the at least one undesirable substance is selected from the group consisting of nicotine, alcohol, medications, pharmaceutical by-products, pharmaceutical metabolites, chemicals, certain bacteria, viruses, and combinations thereof.

13. The kit of claim 1, further comprising instructions, wherein the instructions are selected from at least one of:
(a) instructions for use of the kit;
(b) instructions for delivering at least a portion of the personalized, concentrated human milk composition to the infant; and
(c) instructions for storage of at least a portion of the personalized, concentrated human milk composition for a period of up to five days under refrigerated conditions or for a period of up to six months under frozen conditions.

14. A method of producing a personalized, concentrated human milk composition for an infant, the method comprising the steps of:
(a) obtaining expressed breast milk from a mother of an infant;
(b) analyzing a portion of the expressed breast milk to determine a concentration of at least one nutrient and/or at least one micronutrient present therein;
(c) calculating a desired level of concentration of the expressed breast milk based upon the concentration of the at least one nutrient or micronutrient present in the expressed breast milk and at least one infant factor selected from the group consisting of the infant's height, the infant's weight, the infant's nutritional needs, the infant's fluid volume capacity, the infant's growth curve, and combinations thereof; and
(d) exposing the expressed breast milk to a selective concentration device that concentrates the at least one nutrient and/or the at least one micronutrient analyzed in step (b) to the desired level of concentration calculated in step (c), thereby producing the personalized, concentrated human milk composition.

15. The method of claim 14, further comprising the step of storing at least a portion of the personalized, concentrated human milk composition under refrigerated or frozen conditions for a period of time.

16. The method of claim 14, further comprising the step of administering at least a portion of the personalized, concentrated human milk composition to the infant.

17. The method of claim 14 wherein, in step (b), a concentration of a plurality of nutrients and/or micronutrients present is the expressed breast milk is determined, and wherein, in step (d), the selective concentration device concentrates the plurality of nutrients and/or micronutrients.

18. The method of claim 17, wherein the desired level of concentration calculated in step (c) is based upon a concentration of each of a plurality of nutrients and/or micronutrients present in the expressed breast milk and upon a plurality of infant factors, and wherein the desired level of concentration calculated in step (c) is defined as:
(i) a desired concentration of one or more of the plurality of nutrients and/or micronutrients; and/or
(ii) a desired ratio of two of the nutrients/micronutrients to one another.

19. The method of claim 14, wherein each nutrient is selected from the group consisting of protein, fat, and carbohydrate, and wherein each micronutrient is selected from the group consisting of calcium, phosphorous, potassium, sodium, and chloride.

20. The method of claim 14, further comprising the step of filtering the expressed breast milk to remove at least one undesirable substance from the expressed breast milk.

21. The method of claim 20, wherein the at least one undesirable substance is selected from the group consisting of nicotine, alcohol, medications, pharmaceutical by-products, pharmaceutical metabolites, chemicals, certain bacteria, viruses, and combinations thereof.
